(19) United States
(12) Patent Application Publication
(10) Pub. No.: US 2009/0176856 A1
(43) Pub. Date: Jul. 9, 2009

(54) MUSCARINIC RECEPTOR ANTAGONISTS

(76) Inventors: Anita Mehta, Plainfield, IL (US); Mohammad Salman, Princeton, NJ (US); Pakala Kumara Savithru Sarma, Haryana (IN); Shelly Aeron, Delhi (IN); Anita Chugh, Delhi (IN); Suman Gupta, Haryana (IN)

Correspondence Address: RANBAXY INC., 600 COLLEGE ROAD EAST, SUITE 2100, PRINCETON, NJ, 08540, US

(21) Appl. No.: 11/576,066

(22) PCT Filed: Sep. 26, 2005
(86) PCT No.: PCT/IB2005/002838

§ 371 Date: May 19, 2008

(30) Foreign Application Priority Data
Sep. 27, 2004 (IN) 1849/DEL/2004

(51) Int. Cl. A61K 31/403; C07D 209/52; A61P 13/00; A61P 11/00; A61P 11/06; A61P 3/04; A61P 3/10; C07D 405/08

(52) U.S. Cl. 514/414; 548/515; 514/412; 548/454

(57) ABSTRACT

This present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### FIELD OF THE INVENTION

This present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### BACKGROUND OF THE INVENTION

Muscarinic receptors as members of the G Protein Coupled Receptors (GPCRs) are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented. (for example, the M₁ subtype is located primarily in neuronal tissues such as cerebral cortex and autonomic ganglia, the M₂ subtype is present mainly in the heart where it mediates cholinergically induced bradycardia, and the M₃ subtype is located predominantly on smooth muscle and salivary glands (Nature, 323, p. 411 (1986); Science, 237, p. 527 (1987)).

A review in Current Opinions in Chemical Biology, 3, p. 426 (1999), as well as in Trends in Pharmacological Sciences, 22, p. 409 (2001) by Eglen et. al., describes the biological potentials of modulating muscarinic receptor subtypes by ligands in different disease conditions, such as Alzheimer's Disease, pain, urinary disease condition, chronic obstructive pulmonary disease, and the like.

A review in J. Med. Chem., 43, p. 4333 (2000), by Felder et. al. describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 6, p. 142 (2001).

Birdsall et. al. in Trends in Pharmacological Sciences, 22, p. 215 (2001) have also summarized the recent developments on the role of different muscarinic receptor subtypes using different muscarinic receptor of knock out mice.

Muscarinic agonists such as muscarine and pilocarpine and antagonists such as atropine have been known for over a century, but little progress has been made in the discovery of receptor subtype-selective compounds, making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quarterly derivatives of atropine such as ipratropium bromide are better tolerated than parenterally administered options, but most of these are not ideal anti-cholinergic bronchodilators, due to lack of selectivity for muscarinic receptor sub-types, resulting in dose-limiting side-effects such as thirst, nausea, mydriasis and those associated with the heart such as tachycardia mediated by the M₂ receptor.

Annual Review of Pharmacological Toxicol., 41, p. 691 (2001), describes the pharmacology of the lower urinary tract infections. Although anti-muscarinic agents such as oxybutynin and tolterodine that act non-selectively on muscarinic receptors have been used for many years to treat bladder hyperactivity, the clinical effectiveness of these agents has been limited due to the side effects such as dry mouth, blurred vision and constipation. Tolterodine is considered to be generally better tolerated than oxybutynin. (Steers et. al., in Curr. Opin. Invest. Drugs, 2, 268; Chapple et. al., in Urology, 55, 33; Steers et al., Adult and Pediatric Urology, ed. Gillenwatter et al., pp 1220-1325, St. Louis, Mo.; Mosby. 3^{rd} edition (1996)).

There remains a need for development of new highly selective muscarinic antagonists which can interact with distinct subtypes, thus avoiding the occurrence of adverse effects.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 93/16018 and WO96/33973 are other references of interest; WO 97/45414 are related to 1,4-disubstituted piperidine derivatives; WO 98/05641 describes fluorinated, 1,4-disubstituted piperidine derivatives; U.S. Pat. No. 5,397,800 discloses 1-azabicyclo[2.2.1]heptanes. U.S. Pat. No. 5,001,160 describes 1-aryl-1-hydroxy-1-substituted-3-(4-substituted-1-piperazinyl)-2-propanones. WO 99/43657 describes 2-arylethyl-(piperidin-4-ylmethyl)amine derivatives as muscarinic receptors antagonists. WO 01/090082 describes substituted 1-amino-alkyl lactams and their use as muscarinic receptor antagonists. WO 01/47893 describes azabicycloctane derivatives useful in the treatment of cardiac arrhythmias. WO 01/42213 describes 2-biphenyl-4-piperidinyl ureas. WO 01/42212 describes carbamate derivatives. WO 01/90081 describes amino alkyl lactam. WO 02/53564 describes novel quinuclidine derivatives. WO 02/00652 describes carbamates derived from arylalkyl amines. WO 02/06241 describes 1,2,3,5-tetrahydrobenzo(c)azepin-4-one derivatives. U.S. application No. 20030105071 describes thiazole and other heterocyclic ligands for mammalian dopamine, muscarinic and serotonic receptors and transporters, and method of use thereof. WO 03/033495 describes quinuclidine derivatives and their use as M₂ and/or M₃ muscarinic receptor antagonists. US2003/0171362 describes amino-tetralin derivatives as muscarinic receptor antagonists. US2003/0162780 describes 4-piperidinyl alkyl amine derivatives as muscarinic receptor antagonists. U.S. Pat. No. 5,179,108 disclose derivatives of 4-(aminomethyl)piperidine and their therapeutic applications. WO 03/048125 discloses aminotetralin derivatives as muscarinic receptor antagonists. WO 03/048124 discloses 4-piperidinyl alkylamine derivatives as muscarinic receptor antagonists. WO 2004/052857 and WO 04/004629 disclose 3,6-disubstituted azabicyclo[3.1.0]hexane derivatives useful as muscarinic receptor antagonists. WO 04/005252 discloses azabicyclo derivatives as musacrinic receptor antagonists. discloses WO 04/014853, WO 04/067510 and WO 04/014363 disclose derivatives of 3,6-disubstituted azabicyclohexane useful as muscarinic receptor antagonists. WO 2004/056810 discloses xanthine derivatives as muscarinic receptor antagonists. WO 2004/056811 discloses flaxavate derivatives as muscarinic receptor antagonists. WO 2004/056767 discloses 1-substituted-3-pyrrolidine derivatives as muscarinic receptor antagonists. WO 2004/018422 disclose fluoro and sulphonylamino containing 3,6-disubstituted azabicyclo[3.1.0]hexane derivatives as muscarinic receptor antagonists.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991), describes analogues of oxybutynin, synthesis and antimuscarinic activity of some substituted 7-amino-1-hydroxy-5-heptyn-2-ones and related compounds.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I

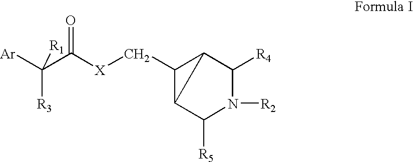

and their pharmaceutically acceptable salts, pharmaceutically acceptable solvates, esters, enantiomers, diastereomers, N-oxides, polymorphs, metabolites, wherein

R₁ can be hydrogen, lower (C₁-C₆)alkyl, lower (C₂-C₇)alkenyl, lower (C₂-C₇)alkynyl, cycloalkyl, amino, substituted amino, —OR_{z} {wherein R_{z} is selected from, for example, hydrogen, —Si(CH₃)₃, lower (C₁-C₆)alkyl, lower (C₂-C₆)alkenyl, lower (C₂-C₆)alkynyl, cycloalkyl, aryl, and —C(═O)NHRᵣ (wherein Rᵣ is selected from, for example, hydrogen, lower (C₁-C₆)alkyl, lower (C₂-C₆)alkenyl, lower (C₂-C₆)alkynyl, aryl, and cycloalkyl)}.
R₂ can be carboxy, —SO₂R₆ {wherein R₆ is selected from, for example, alkyl, alkenyl, alkynyl, cycloalkyl, —NRₚR_{q} (wherein Rₚ and R_{q} are selected from, for example, hydrogen, alkyl, alkenyl, alkynyl, cycloalkyl, aryl, aralkyl, heterocyclyl, heteroaryl, heterocyclylalkyl, and heteroarylalkyl), aryl, aralkyl, heteroaryl, heterocyclyl, heterocyclylalkyl, and heteroarylalkyl, or Rₚ and R_{q} may also together join to form a heterocyclyl ring}, —C(═O)OR₇ (wherein R₇ is selected from, for example, alkyl, alkenyl, alkynyl, cycloalkyl, aryl, and aralkyl), —C(═O)NRₓR_{y} {wherein Rₓ and R_{y} are each independently selected from, for example, hydrogen, hydroxy (as restricted by the definition that both Rₓ and R_{y} cannot be hydroxy at the same time), alkyl, alkenyl, alkynyl, aryl, aralkyl, S(O)₂R₆ wherein R₆ is the same as defined above, heteroaryl, heterocyclyl, heteroarylalkyl, and heterocyclylalkyl, or Rₓ and R_{y} may also together join to form a heterocyclyl ring}, acyl, halogen (F, Cl, Br, I), cyano, —NRₓR_{y}, wherein Rₓ and R_{y} are the same as defined above), or —C(═O)CH₂ORₓ, (wherein Rₓ is the same as defined above).
R₃ can be alkyl, alkenyl, alkynyl, cycloalkyl, aryl, aralkyl, heterocyclyl, heteroaryl, heterocyclylalkyl, and heteroarylalkyl.
R₄ and R₅ can be independently selected from, for example, hydrogen, lower (C₁-C₆) alkyl, lower (C₂-C₇)alkenyl, and lower (C₂-C₇)alkynyl.
X can be oxygen, —NR₇ (wherein R₇ is selected from, for example, hydrogen, lower (C₁-C₆)alkyl, lower (C₂-C₇)alkenyl, lower (C₂-C₇)alkynyl, aralkyl, and aryl.
Ar can be aryl, heteroaryl, and heterocyclyl.

In accordance with a further aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with another aspect, there is provided a method for treatment or prophylaxis of an animal or a human suffering from a disease or disorder associated with muscarinic receptors, comprising administering to a patient in need thereof, an effective amount of a muscarinic receptor antagonist compound as described above.

In accordance with yet a further aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with yet another aspect, there are provided processes for preparing the compounds as described above.

The compounds described herein exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₃ receptors. Therefore, pharmaceutical compositions for the possible treatment for the disease or disorders associated with muscarinic receptors are provided. In addition, the compounds can be administered orally or parenterally.

The following definitions apply to terms as used herein

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q}, —C(═O)heteroaryl, C(═O)heterocyclyl, —O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}N—HC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “alkylene,” as used herein, refers to a diradical branched or unbranched saturated hydrocarbon chain having from 1 to 6 carbon atoms and one or more hydrogen can optionally be substituted with alkyl, hydroxy, halogen or oximes. This term can be exemplified by groups such as methylene, ethylene, propylene isomers (e.g., —CH₂CH₂CH₂ and —CH(CH₃)CH₂) and the like. Alkylene may further be substituted with one or more substituents such as alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR₂ (wherein R₂ is the same as defined earlier), —NHC(═O)Rₓ, —NRₓR_{y}, —C(═O)NRₓR_{y}, —NHC(═O)NRₓR_{y}, —C(═O)heteroaryl, C(═O)heterocyclyl, —O—C(═O)NRₓR_{y} (wherein Rₓ and R_{y} are the same as defined earlier), nitro, —S(O)ₙR₃ (wherein n and R₃ are the same as defined earlier). Unless otherwise constrained by the definition, all substituents may be further substituted by 1-3 substituents chosen from alkyl, carboxy, —COOR₂ (wherein R₂ is the same as defined earlier), —NRₓR_{y}, —C(═O)NRₓR_{y}, —OC(═O)NRₓR_{y}, —NHC(═O)NRₓR_{y} (wherein Rₓ and R_{y} are the same as defined earlier), hydroxy, alkoxy, halogen, CF₃, cyano, and —S(O)ₙR₃ (where R₃ and n are the same as defined earlier). Alkylene can also be optionally interrupted by 1-5 atoms of groups independently chosen from oxygen, sulfur and —NRₐ, where Rₐ is chosen from hydrogen, alkyl, cycloalkyl, alkenyl, alkynyl, aryl, acyl, aralkyl, —C(═O)OR₂ (wherein R₂ is the same as defined earlier), —S(O)ₙR₃ (where n and R₃ are the same as defined earlier), —C(═O)NRₓR_{y} (wherein Rₓ and R_{y} are as defined earlier) —CONH—, —C═O or —C═NOH.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or SO₂R₆ (wherein R₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (where R₆ is same as defined earlier).

The term “alkenylene” unless otherwise specified, refers to a diradical of a branched or unbranched unsaturated hydrocarbon group preferably having from 2 to 6 carbon atoms with cis, trans or geminal geometry. In the event that alkenylene is attached to the heteroatom, the double bond cannot be alpha to the heteroatom. The alkenylene group can be connected by two bonds to the rest of the structure of compound of Formula I. Alkenylene may further be substituted with one or more substituents such as alkyl, alkynyl, alkoxy, cycloalkyl, acyl, acylamino, acyloxy, —NHC(═O)Rₓ, —NRₓR_{y}, —C(═O)NRₓR_{y}, —NHC(═O)NRₓR_{y}, —OC(═O)NRₓR_{y} (wherein Rₓ, and R_{y} are the same as defined earlier), alkoxycarbonylamino, azido, cyano, halogen, hydroxy, oxo, thiocarbonyl, carboxy, —COOR₂ (wherein R₂ is the same as defined earlier), arylthio, thiol, alkylthio, aryl, aralkyl, aryloxy, heterocyclyl, heteroaryl, heterocyclyl alkyl, heteroaryl alkyl, aminosulfonyl, alkoxyamino, nitro, —S(O)ₙR₃ (where R₃ and n are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, cyano, —NRₓR_{y}, —C(═O)NRₓR_{y}, —OC(═O)NRₓR_{y} (wherein Rₓ and R_{y} are the same as defined earlier) and —S(O)ₙR₃ (where R₃ and n are the same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, —C(═O)NR_{f}R_{q} (wherein R_{f} and R_{q} are the same as defined earlier), cyano, or —SO₂R₆ (where R₆ is same as defined earlier).

The term “alkynylene” unless otherwise specified, refers to a diradical of a triply-unsaturated hydrocarbon, preferably having from 2 to 6 carbon atoms. In the event that alkynylene is attached to the heteroatom, the triple bond cannot be alpha to the heteroatom. The alkenylene group can be connected by two bonds to the rest of the structure of compound of Formula I. Alkynylene may further be substituted with one or more substituents such as alkyl, alkenyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, nitro, heterocyclyl, heteroaryl, heterocyclyl alkyl, heteroarylalkyl, —NHC(═O)Rₓ, —NRₓR_{y}, —NHC(═O)NRₓR_{y}, —C(═O)NRₓR_{y}, —OC(═O)NRₓR_{y} (wherein Rₓ, and R_{y} are the same as defined earlier), —S(O)ₙR₃ (where R₃ and n are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, CF₃, —NRₓR_{y}, —C(═O)NRₓR_{y}, —NHC(═O)NRₓR_{y}, —C(═O)NRₓR_{y} (wherein Rₓ and R_{y} are the same as defined earlier), cyano, and —S(O)ₙR₃ (where R₃ and n are the same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O) R_{f}, —C(═O) NR_{f}R_{q}, —O—C(═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —OC(═O)NR_{f}R_{q} (wherein R_{f} and R_{q} are the same as defined earlier), cyano or —SO₂R₆ (where R₆ is same as defined earlier). “Cycloalkylalkyl” refers to alkyl-cycloalkyl group linked through alkyl portion, wherein the alkyl and cycloalkyl are the same as defined earlier.

The term “alkoxy” denotes the group O-alkyl, wherein alkyl is the same as defined above.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, C(═O)NR_{f}R_{q}, —NHC(═O)NR_{f}R_{q}, —O—C(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term “aralkenyl,” unless otherwise specified, refers to alkenyl-aryl linked through alkenyl (wherein alkenyl is as defined above) portion and the alkenyl portion contains 1 to 6 carbon atoms and aryl is as defined below.

The term “aryloxy” denotes the group O-aryl, wherein aryl is as defined above.

The term “carboxy,” as defined herein, refers to —C(═O)OH.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z}, or —NHOH}, —C(═O)NR_{f}R_{q} and —NHC(═O)NR_{f}R_{q}, —SO₂R₆, —O—C(═O)NR_{f}R_{q}, —O—C(═O)R_{f}, —O—C(═O)OR_{f} (wherein R₆, R_{f} and R_{q} are as defined earlier, and R_{z} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, alkaryl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Alkylcarbonyl” refers to —C(═O)R″, wherein R″ is selected from alkyl, cycloalkyl, aryl, aralkyl, heteroaryl, heterocyclyl, heteroarylalkyl or heterocyclylalkyl.

“Alkylcarboxy” refers to —O—C(═O)R″, wherein R″ is selected from alkyl, cycloalkyl, aryl, aralkyl, heteroaryl, heterocyclyl, heteroarylalkyl or heterocyclylalkyl.

“Amine,” unless otherwise specified, refers to —NH₂. “Substituted amine,” unless otherwise specified, refers to —N(Rₖ)₂, wherein each Rₖ independently is selected from hydrogen {provided that both Rₖ groups are not hydrogen (defined as “amino”)}, alkyl, alkenyl, alkynyl, aralkyl, cycloalkyl, aryl, heteroaryl, heterocyclyl, heterocyclylalkyl, heteroarylalkyl, acyl, SO₂R₆ (wherein R₆ is as defined above), —C(═O)NR_{f}R_{q}, NHC(═O)NR_{f}R_{q}, or —NHC(═O)OR_{f} (wherein R_{f} and R_{q} are as defined earlier).

“Amine,” unless otherwise specified, refers to —NH₂. “Substituted amino” unless otherwise specified, refers to a group —N(Rₖ)₂ wherein each Rₖ is independently selected from the group hydrogen provided that both Rₖ groups are not hydrogen (defined as “amino”), alkyl, alkenyl, alkynyl, aralkyl, cycloalkyl, aryl, heteroaryl, heterocyclyl, heterocyclylalkyl, heteroarylalkyl, acyl, S(O)ₘR₆ (wherein m and R₆ is the same as defined above), —C(═Rᵥ)NRₓR_{y} (wherein Rᵥ is O or S & Rₓ, and R_{y} are the same as defined earlier) or NHC(═Rᵥ)NR_{y}Rₓ, (wherein Rᵥ, R_{y} and Rₓ are the same as defined earlier). Unless otherwise constrained by the definition, all amino substituents may optionally be further substituted by 1-3 substituents chosen from alkyl, aralkyl, cycloalkyl, aryl, heteroaryl, heterocyclyl, carboxy, —COOR₇ (wherein R₇ is the same as defined earlier), hydroxy, alkoxy, halogen, CF₃, cyano, —C(═Rᵥ)NRₓR_{y} (wherein Rᵥ is the same as defined earlier), —O(C═O)NRₓR_{y}, —OC(═Rᵥ)NRₓR_{y} (wherein Rₓ, R_{y} and Rᵥ are the same as defined earlier), —S(O)ₘR (where R₆ and m is the same as defined above).

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The compounds of this invention contain one or more asymmetric carbon atoms and thus can occur as racemates and racemic mixtures, single enantiomers, diastereomeric mixtures and individual diastereomers. All such isomeric forms of these compounds are expressly included in the present invention. Each stereogenic carbon may be of the R or S configuration. Although the specific compounds exemplified in this application may be depicted in a particular stereochemical configuration, compounds having either the opposite stereochemistry at any given chiral center or mixture(s) thereof are envisioned as part of the invention. Although amino acids and amino acid side chains may be depicted in a particular configuration, both natural and unnatural forms are envisioned as part of the invention.

### DETAILED DESCRIPTION OF THE INVENTION

The compounds of the present invention may be prepared by techniques well known in the art and familiar to the average synthetic organic chemist. In addition, the compounds of the present invention may be prepared by the following the reaction Schemes I, II and III

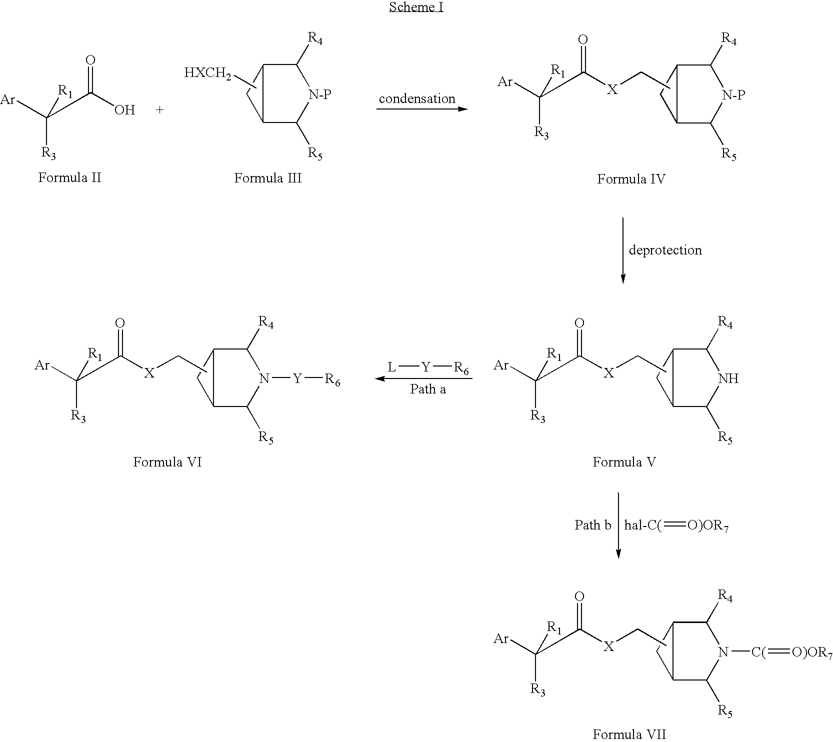

The compounds of Formulae VI and VII may be prepared according to Scheme I. Thus, the preparation comprises condensing a compound of Formula II (wherein Ar, R₁ and R₃ are the same as defined earlier) with a compound of Formula III (wherein X, R₄ and R₅ are the same as defined earlier and P is a protecting group for example, aralkyl or acyl) to give a compound of Formula IV, which can be deprotected to give a compound of Formula V,

Path a: the compound of Formula V is reacted with a compound of Formula L-Y—R₆ (wherein L is a leaving group for example halogen (F, Cl, Br, I), Y is —C(═O), SO₂ and R₆ is the same as defined earlier) to give a compound of Formula VI.
Path b: the compound of Formula V is reacted with a compound of Formula hal-C(═O)OR₇ (wherein R₇ is the same as defined earlier and hal is halogen (Br, Cl, I)) to give a compound of Formula VII.

The condensation of a compound of Formula II with a compound of Formula III can be carried out in the presence of a condensing agent (for example, 1-(3-dimethylaminopropyl)-3-ethyl carbodiimide hydrochloride or dicyclohexylcarbodiimide in an organic base (for example, 1,8-diazabicyclo[5.4.0]undec-7-ene, N-methylmorpholine, triethylamine, diisopropylethylamine or pyridine) in an organic solvent (for example, N,N-dimethylformamide, chloroform, tetrahydrofuran, dioxane, diethylether, benzene or toluene) to give a compound of Formula IV which on deprotection (for example, hydrogenatically utilizing palladium on carbon under catalytic hydrogenation transfer conditions of ammonium formate and palladium on carbon) in an organic solvent (for example, methanol, ethanol, tetrahydrofuran and acetonitrile) gives a compound of Formula V, which on reaction with a compound of Formula L-Y—Rᵣ (Path a) in the presence of a base (for example, triethylamine, diisopropylethylamine or pyridine) in an organic solvent for example, dichloromethane, dichloroethane, chloroform or carbon tetrachloride) gives a compound of Formula VI. The reaction of a compound of Formula V (Path b) with a compound of Formula hal-C(═O)OR₇ can be carried out in the presence of a base (for example, triethylamine, diisopropylethylamine or pyridine) in an organic solvent (for example, dichloromethane, dichloroethane, chloroform or carbon tetrachloride) to give a compound of Formula VII.

Particular illustrative compounds which can be prepared following Scheme I include those listed below (also shown in Table I and II):
N-{[(1α,5α,6α)-3-(4-Nitrobenzenesulphonyl)-3-azabicyclo[3.1.0]hex-6-ylmethyl]}-2-cyclopentyl-2-hydroxy-2-phenyl acetamide (Compound No. 4),
N-{[(1α,5α,6α)-3-Benzenesulfonyl-3-azabicyclo[3.1.0]hex-6-ylmethyl]}-2-cyclopentyl-2-hydroxy-2-phenyl acetamide (Compound No. 5),
N-{[(1α,5α,6α)-3-(3,5-Dinitrobenzoyl)-3-azabicyclo[3.1.0]hex-6-ylmethyl]}-2-cyclopentyl-2-hydroxy-2-phenyl acetamide (Compound No. 6),
N-{[(1α,5α,6α)-3-(2-Benzyloxyacetyl)-3-azabicyclo[3.1.0]hex-6-ylmethyl]}-2-cyclopentyl-2-hydroxy-2-phenyl acetamide (Compound No. 7),
N-{[(1α,5α,6α)-3-Benzoyl-3-azabicyclo[3.1.0]hex-6-ylmethyl]}-2-hydroxy-2-cyclopentyl-2-phenyl acetamide (Compound No. 8),
N-{[(1α,5α,6α)-3-(3-Nitrobenzenesulphonyl)-3-azabicyclo[3.1.0]hex-6-ylmethyl]}-2-hydroxy-2-cyclopentyl-2-phenyl acetamide (Compound No. 9),
N-{[(1α,5α,6α)-3-(2-Benzo[1,3]dioxol-5-yl-acetyl)-3-azabicyclo[3.1.0]hex-6-ylmethyl]}-2-hydroxy-2-cyclopentyl-2-phenyl acetamide (Compound No. 10),
N-{[(1α,5α,6α)-3-(4-Trifluoromethylbenzenesulfonyl)-3-azabicyclo[3.1.0]hex-6-ylmethyl]}-2-cyclopentyl-2-hydroxy-2-phenyl acetamide (Compound No. 11),
N-{[(1α,5α,6α)-3-[2-(3,5-Difluoro-phenyl)-acetyl]-3-azabicyclo[3.1.0]hex-6-ylmethyl]}-2-hydroxy-2-cyclopentyl-2-phenyl acetamide (Compound No. 13),
N-{[(1α,5α,6α)-3-(4-Tert-butylbenzenesulfonyl)-3-azabicyclo[3.1.0]hex-6-ylmethyl]}-2-cyclopentyl-2-hydroxy-2-phenyl acetamide (Compound No. 14),
N-{[(1α,5α,6α)-3-(2-Fluorobenzoyl)-3-azabicyclo[3.1.0]hex-6-ylmethyl}]-2-cyclopentyl-2-hydroxy-2-phenyl acetamide (Compound No. 15),
N-{[(1α,5α,6α)-3-(3,4,5-Trimethoxybenzoyl)-3-azabicyclo[3.1.0]hex-6-yl methyl]}-2-cyclopentyl-2-hydroxy-2-phenyl acetamide (Compound No. 16),
N-{[(1α,5α,6α)-3-Phenylacetyl-3-azabicyclo[3.1.0]hex-6-ylmethyl]}-2-cyclopentyl-2-hydroxy-2-phenyl acetamide (Compound No. 18),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-4-nitro-benzyl ester (Compound No. 19),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid isobutyl ester (Compound No. 20),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid 4-nitro-phenyl ester (Compound No. 21),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid benzyl ester (Compound No. 22),
N-{[(1α,5α,6α)-3-(4-Fluorobenzenesulphonyl)-3-azabicyclo[3.1.0]hex-6-yl methyl}]-2-cyclopentyl-2-hydroxy-2-phenyl acetamide (Compound No. 23)
N-{[(1α,5α,6α)-3-(2,4,6-Trisopropylbenzenesulphonyl)-3-azabicyclo[3.1.0]hex-6-yl methyl]}-2-cyclopentyl-2-hydroxy-2-phenyl acetamide (Compound No. 24)
N-{[(1α,5α,6α)-3-(3,5-Dimethylbenzoyl)-3-azabicyclo[3.1.0]hex-6-ylmethyl]}-2-cyclopentyl-2-hydroxy-2-phenyl acetamide (Compound No. 27)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid 9H-fluoren-9-ylmethyl ester (Compound No. 35)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid butyl ester (Compound No. 36)
N-{[(1α,5α,6α)-3-(Methanesulphonyl)-3-azabicyclo[3.1.0]hex-6-ylmethyl]}-2-cyclopentyl-2-hydroxy-2-phenyl acetamide (Compound No. 37)
N-{[(1α,5α,6α)-3-(4-Methoxybenzoyl)-3-azabicyclo[3.1.0]hex-6-ylmethyl]}-2-cyclopentyl-2-hydroxy-2-phenyl acetamide (Compound No. 39)
N-{[(1α,5α,6α)-3-(3-Benzo[1,3]-dioxol-5-yl-propionyl)-3-azabicyclo[3.1.0]hex-6-yl methyl]}-2-cyclopentyl-2-hydroxy-2-phenyl acetamide (Compound No. 40)
N-{[(1α,5α,6α)-3-(Dimethylsulfamoyl)-3-azabicyclo[3.1.0]hex-6-yl methyl]}-2-cyclopentyl-2-hydroxy-2-phenyl acetamide (Compound No. 41)
   their pharmaceutically acceptable salts, pharmaceutically acceptable solvates, stereoisomers or polymorphs.

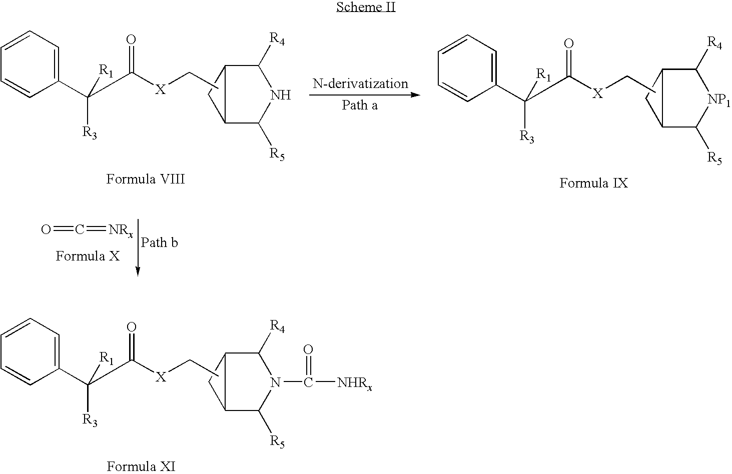

The compounds of Formulae IX and XI may be prepared according to Scheme II. Thus,
Path a: the compound of Formula VIII (wherein X, R₁, R₃, R₄ and R₅ are the same as defined earlier) undergoes N-derivatization to give a compound of Formula IX [wherein P₁ is halogen (F, Cl, Br or I), cyano or —C(═O)OR₇ (R₇ is the same as defined earlier)].
Path b: the compound of Formula VIII is reacted with a compound of Formula X (wherein Rₓ is the same as defined earlier) to give a compound of Formula XI.

The N-derivatization of a compound of Formula VIII (Path a) (when P₁ is halogen) can be carried out with halogenating agent (for example, sodium hypochlorite, sodium hypobromite or sodium hypoiodite) in an organic solvent (for example, dichloromethane, dichloroethane, chloroform or carbon tetrachloride) to give a compound of Formula IX. The N-derivatization of a compound of Formula VIII (when P₁ is cyano) can be carried out with a nitrilating agent (for example, cyanogen bromide) in the presence of an organic base (for example, triethylamine, diisopropylethylamine or pyridine) in an organic solvent (for example, dichloromethane, dichloroethane, chloroform or carbon tetrachloride) to give a compound of Formula IX. The N-derivatization of a compound of Formula VIII (when P₁ is —C(═O)OR₇) can be carried out with anhydrides (for example, ditert-butoxycarbonyl anhydride, dipropoxycarbonyl anhydride, dimethoxycarbonyl anhydride or diethoxycarbonyl anhydride) in the presence of an organic base (for example, triethylamine, diisopropylethylamine or pyridine) in an organic solvent (for example, dichloromethane, dichloroethane, chloroform or carbon tetrachloride) to give a compound of Formula IX. The compound of Formula VIII (Path b) can be reacted with an isocyanate of Formula X in an organic solvent (for example, dichloroethane, dichloromethane, chloroform or carbon tetrachloride) to give a substituted urea of Formula XI.

Alternatively, the compound of Formula XI can also be prepared by reacting a compound of Formula VIII with an appropriate amine in the presence of carbonyldiimidazole (CDI) or with carbamates such as phenyl carbamate or p-nitrophenyl carbamate.

Particular representative compounds which can be prepared following Scheme II include those listed below (also listed in Table I):
N-{[(1α,5α,6α)-6-{[(2-Cyclopentyl-2-hydroxy-2-phenyl-acetylamino)-methyl]-3-aza-bicyclo[3.1.0]hexane-3-carboxylic acid tert-butyl ester (Compound No. 1)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid benzylamide (Compound No. 3)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (4-trifluoromethyl-phenyl)-amide (Compound No. 12)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (4-fluorophenyl)-amide (Compound No. 17)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid allylamide (Compound No. 25)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (2,4-dimethoxy-phenyl)-amide (Compound No. 26)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (4-benzyloxy-phenyl)-amide (Compound No. 28)
N-{[(1α,5α,6α)-3-Chloro-3-azabicyclo[3.1.0]hex-6-ylmethyl]}-2-cyclopentyl-2-hydroxy-2-phenyl acetamide (Compound No. 29)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid amide (Compound No. 30)
N-{[(1α,5α,6α)-3-Cyano-3-azabicyclo[3.1.0]hex-6-ylmethyl]}-2-cyclopentyl-2-hydroxy-2-phenyl acetamide (Compound No. 31)
N-{[(1α,5α,6α)-3-Chloro-3-azabicyclo[3.1.0]hex-6-ylmethyl]}-2-cyclopentyl-2-hydroxy-2-phenyl acetamide hydrochloride salts (Compound No. 32)
N-{[(1α,5α,6α)-3-Chloro-3-azabicyclo[3.1.0]hex-6-ylmethyl]}-2-cyclohexyl-2-hydroxy-2-phenyl acetamide (Compound No. 33)
N-{[(1α,5α,6α)-3-Chloro-3-azabicyclo[3.1.0]hex-6-ylmethyl]}-2-hydroxy-2-diphenyl acetamide (Compound No. 34)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (2,4-difluoro-phenyl)-amide (Compound No. 38)
   their pharmaceutically acceptable salts, pharmaceutically acceptable solvates, stereoisomers or polymorphs.

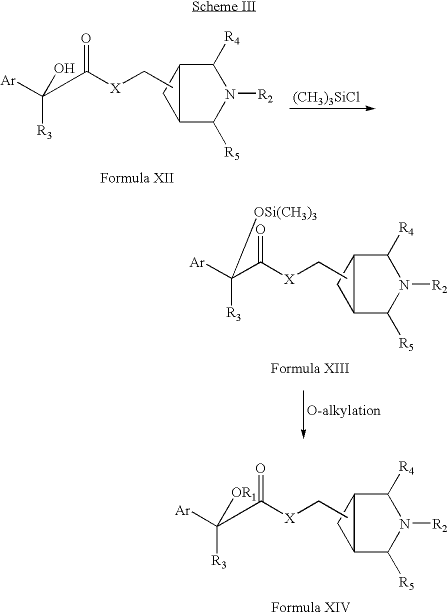

The compound of Formula XIV may be prepared by following Scheme III. The preparation comprises reacting a compound of Formula XII (where in X, R₂, R₃, R₄ and R₅ are the same as defined earlier) with trimethyl silyl chloride to give a compound of Formula XIII, which undergoes O-alkylation to give a compound of Formula XIV (wherein Rₜ is alkyl).

The reaction of a compound of Formula XII with trimethyl silyl chloride can be carried out in an organic base (for example, imidazole, triethylamine, N-methylmorpholine, diisopropylethylamine or pyridine) in an organic solvent (for example, dimethylformamide, tetrahydrofuran, dioxane or diethylether) to give a compound of Formula XIII which can undergo O-alkylation in the presence an organic base (for example, sodium hydride or sodium cyanoboro hydride) in an organic solvent (for example, tetrahydrofuran, dimethylformamide, diethylether or dioxane) to give a compound of Formula XIV.

Particular representative compounds which may be prepared following Scheme III include those listed below (also listed in Table I):
N-{[(1α,5α,6α)-3-Terbutyl-carboxy-3-azabicyclo[3.1.0]hex-6-ylmethyl]}-2-cyclopentyl-2-methoxy-2-phenyl acetamide (Compound No. 2)
   their pharmaceutically acceptable salts, pharmaceutically acceptable solvates, stereoisomers or polymorphs.

Also, in all the above representative examples wherever amines are specified, one skilled in an art would optionally convert them to their respective salts, for example amines can be converted to corresponding hydrochloride salts with ethanolic hydrochloric acid solution in an organic solvent selected from the group consisting of dichloromethane, dichloroethane, chloroform or carbon tetrachloride.

In the above schemes, where specific bases, solvents, condensing agents, etc. are mentioned, it is to be understood that other acids, bases, solvents, condensing agents, hydrolyzing agents, etc, known to those skilled in an art may also be used. Similarly the reaction temperature and duration of the reactions may be adjusted according to desired needs.

TABLE I

| | |
|---|---|
| | Formula I |
| 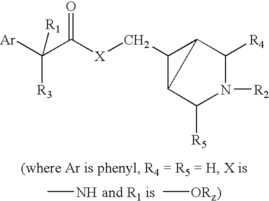 | |

| Compound | | | |
|---|---|---|---|
| No. | R_{z} | R₂ | R3 |
| 1 | H | —C(═O)OC(CH₃)₃ | 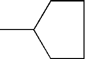 |
| 2 | —CH₃ | —C(═O)OC(CH₃)₃ | 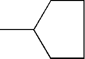 |
| 3 | H | —C(═O)NHCH₂Ph | 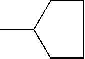 |
| 4 | H | 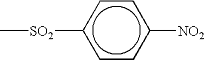 | 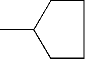 |
| 5 | H | 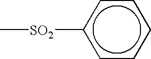 | 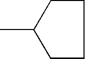 |
| 6 | H | 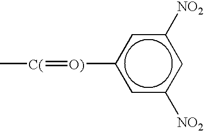 | 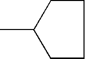 |
| 7 | H | —C(═O)CH₂O—CH₂Ph | 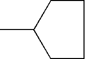 |
| 8 | H | 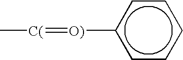 | 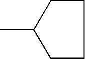 |
| 9 | H | 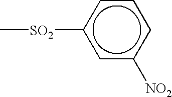 | 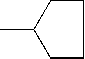 |
| 10 | H | 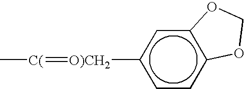 | 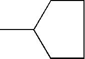 |
| 11 | H | 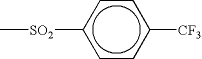 | 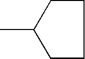 |
| 12 | H | 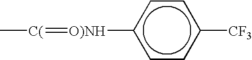 | 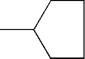 |
| 13 | H | 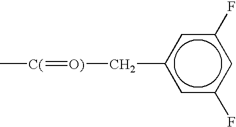 | 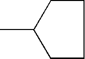 |
| 14 | H | 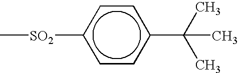 | 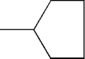 |
| 15 | H | 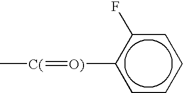 | 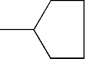 |
| 16 | H | 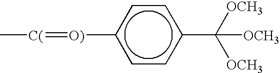 | 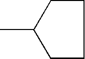 |
| 17 | H | 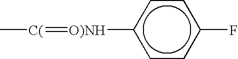 | 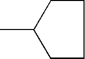 |
| 18 | H | 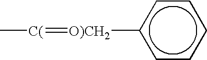 | 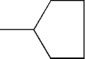 |
| 19 | H | 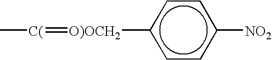 | 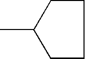 |
| 20 | H | —C(═O)OCH₂—CH(CH₃)₂ | 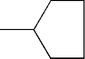 |
| 21 | H | 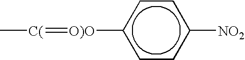 | 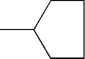 |
| 22 | H | —C(═O)O—CH₂Ph | 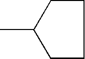 |
| 23 | H | 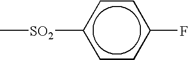 | 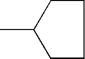 |
| 24 | H | 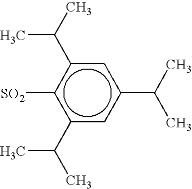 | 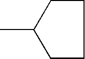 |
| 25 | H | 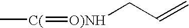 | 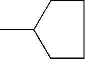 |
| 26 | H | 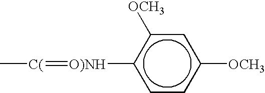 | 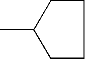 |
| 27 | H | 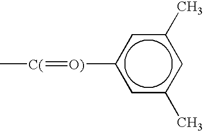 | 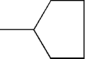 |
| 28 | H | 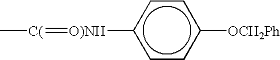 | 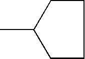 |
| 29 | H | —Cl | 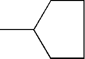 |
| 30 | H | 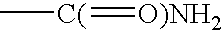 | 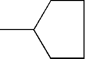 |
| 31 | H | —CN | 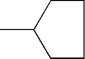 |
| 32* | H | Cl | 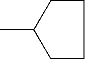 |
| 33 | H | —Cl | 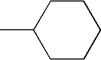 |
| 34 | H | Cl | 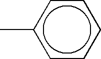 |
| 35 | H | 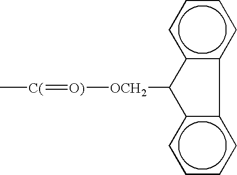 | 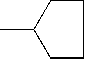 |
| 36 | H | —C(═O)O(CH₂)₃CH₃ | 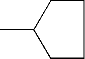 |
| 37 | H | —SO₂CH₃ | 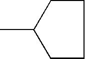 |
| 38 | H | 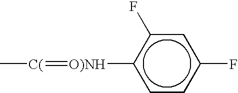 | 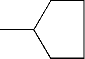 |

* refers to hydrochloride salt of compound No. 29.

TABLE II

| | |
|---|---|
| | Formula I |
| 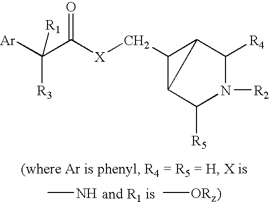 | |

| Com- | | | |
|---|---|---|---|
| pound | | | |
| No. | R_{z} | R₂ | R3 |
| 39 | H | 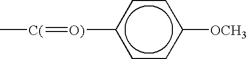 | 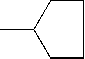 |
| 40 | H | 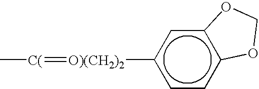 | 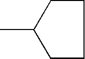 |

Because of their valuable pharmacological properties, the compounds described herein may be administered to an animal for treatment orally, or by a parenteral route.

The pharmaceutical compositions described herein can be produced and administered in dosage units, each unit containing a certain amount of at least one compound described herein and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

### EXAMPLES

Various solvents, such as acetone, methanol, pyridine, ether, tetrahydrofuran, hexanes, and dichloromethane, were dried using various drying reagents according to procedures described in literature. IR Spectra were recorded as Nujol Mulls or a thin neat film on a Perkin Elmer Paragon instrument, Nuclear Magnetic Resonance (NMR) were recorded on Varian XL-300 MHz instrument using tetramethylsilane as an internal standard.

### Synthesis of (1α,5α,6α)-6-aminomethyl-3-benzyl-3-azabicyclo[3.1.0]hexane

This compound was synthesised following the procedure described in EP 0413455 A2.

### Synthesis of 2-hydroxy-2-cyclopentyl-phenyl Acetic Acid

The compound was synthesised following the procedure described in Kadin et al., J. Org. Chem., 1962, 27, 240-245.

### Synthesis of N-(1α,5α,6α)-(3-benzyl-3-azabicyclo[3.1.0]hex-6-ylmethyl)-2-cyclopentyl 2-hydroxy-2-phenyl-acetamide

### Step a: Synthesis of 2-hydroxy-2-cyclopentyl-2-phenyl Acetic Acid

This was prepared following the procedure described in J. Amer. Chem. Soc., 75, 265 (1953).

### Step b: Synthesis of (1α,5α,6α)-6-aminomethyl-3-benzyl-3-azabicyclo[3.1.0]hexane.

The compound was prepared following the procedure described in EP 0 413 455 A2.

### Step c: Synthesis of (1α,5α,6α)-N-(3-benzyl-3-azabicyclo[3.1.0]hex-6-ylmethyl)-2-cyclopentyl-2-hydroxy-2-phenyl Acetamide

To a solution of a compound obtained from step b above (29.9 mmole, 6.05 g) in dimethylformamide (100 ml) was added 2-hydroxy-2-cyclopentyl-2-phenyl acetic acid (commercially available) (27.2 mmole, 6.0 g) followed by the addition of 1-(3-dimethylaminopropyl)-3-ethyl carbodiimide and cooled at 0° C. The reaction mixture was treated with hydroxy benzotriazole (29.9 mmole, 4.04 gm) followed by addition of N-methyl morpholine (54.4 mmole, 5.2 g) and was stirred at 0° C. for 1 hour and at room temperature overnight. The reaction mixture was poured into saturated sodium bicarbonate solution. The organic compound was extracted with ethyl acetate. The organic layers were washed with water and dried over anhydrous sodium sulphate and concentrated under reduced pressure. The residue was purified by column chromatography to yield the title compound with 95% yield.

The analogs of (1α,5α,6α)-N-(3-benzyl-3-azabicyclo[3.1.0]hex-6-ylmethyl)-2-cyclopentyl-2-hydroxy-2-phenyl-acetamide described below, can be prepared by replacing appropriate acid in place of 2-hydroxy 2-cyclopentyl phenyl acetic acid.
N-(1α,5α,6α)-(3-Benzyl-3-azabicyclo[3.1.0]hex-6-ylmethyl)-2-cyclobutyl-2-hydroxy-2-phenyl acetamide
N-(1α,5α,6α)-(3-Benzyl-3-azabicyclo[3.1.0]hex-6-ylmethyl)-2-cyclohexyl-2-hydroxy-2-phenyl acetamide
N-(1α,5α,6α)-(3-Benzyl-3-azabicyclo[3.1.0]hex-6-ylmethyl)-2-hydroxy-2,2-phenyl acetamide

### Synthesis of N-(1α,5α,6α)-(3-azabicyclo[3.1.0]hex-6-ylmethyl)-2-cyclopentyl-2-hydroxy-2-phenyl Acetamide

To a solution of N-(1α,5α,6α)-(3-benzyl-3-azabicyclo[3.1.0]hex-6-ylmethyl)-2-cyclopentyl-2-hydroxy-2-phenyl acetamide (1.0 g, 2.48 mmole) in dry methanol (25.0 ml), was added palladium on carbon (5%, 0.2 g) under N₂ atmosphere followed by the addition of ammonium formate (0.8 g, 12.38 mmole) under constant stirring. The reaction mixture was refluxed for half an hour under N₂ atmosphere. The reaction mixture was cooled to room temperature and the reaction mixture was filtered through hyflo bed. The hyflo bed was washed with methanol (75.0 ml), ethyl acetate (25.0 ml) and water (25.0 ml). The filtrate was concentrated under vacuum. The residue thus obtained was diluted with water and pH of the resulting solution was adjusted to pH˜14 with sodium hydroxide. The compound was extracted with ethyl acetate (2×50 ml) and the ethyl acetate layer was washed with water and brine solution. The layer was dried over anhydrous sodium sulphate and concentrated to give the title compound with 96.2% yield.

The analogs of N-(1α,5α,6α)-(3-azabicyclo[3.1.0]hex-6-ylmethyl)-2-cyclopentyl-2-hydroxy-2-phenyl acetamide described below, can be prepared by deprotection of appropriate amine, as applicable in each case.
N-(1α,5α,6α)-(3-Azabicyclo[3.1.0]hex-6-ylmethyl)-2-cyclobutyl-2-hydroxy-2-phenyl acetamide
N-(1α,5α,6α)-(3-Azabicyclo[3.1.0]hex-6-ylmethyl)-2-cyclohexyl-2-hydroxy-2-phenyl acetamide
N-(1α,5α,6α)-(3-Azabicyclo[3.1.0]hex-6-ylmethyl)-2-hydroxy-2,2-diphenyl acetamide

### Scheme I, Path A Procedure

### Example 1

### Synthesis of N-}[(1α,5α,6α)-3-(4-nitrobenzenesulphonyl)-3-azabicyclo[3.1.0]hex-6-yl methyl]}-2-cyclopentyl-2-hydroxy-2-phenyl Acetamide (Compound No. 4)

To a solution of N-(1α,5α,6α)-(3-azabicyclo[3.1.0]hex-6-ylmethyl)-2-cyclopentyl-2-hydroxy-2-phenyl acetamide (0.796 mmole) and triethyl amine (1.592 mmol) in dichloromethane (10.0 ml) at 0° C. was added p-nitro phenyl sulphonyl chloride (0.955 mmole). The reaction mixture was stirred for 1 hour at 0° C. and then at room temperature for overnight. The solid thus separated was filtered, washed thoroughly with dichloromethane and dried to get the title compound with 60% yield; M.P: 225.3-227.1° C.; IR (KBr): 1642.5 cm⁻¹; ¹H NMR (DMSO-d₆): δ 8.39-8.42 (m, 2H), 7.91-7.99 (m, 2H), 7.54-7.56 (m, 2H), 7.22-7.32 (m, 3H), 5.47 (s, 1H), 3.06-3.09 (m, 3H), 2.90-2.92 (m, 2H), 2.76-2.79 (m, 2H), 1.42-1.47 (m, 9H), 1.25-1.28 (m, 2H), 0.61 (brs, 1H); Mass (m/z): 500 (M⁺+1), 482 (M⁺-OH).

Analogs of N-{[(1α,5α,6α)-3-(4-nitrobenzenesulphonyl)-3-azabicyclo[3.1.0]hex-6-yl methyl]}-2-cyclopentyl-2-hydroxy-2-phenyl acetamide (Compound No. 4) described below, can be prepared by replacing appropriate sulfonyl group in place of n-nitro phenyl sulfonyl chloride, as applicable in each case.
N-{[(1α,5α,6α)-3-Benzenesulfonyl-3-azabicyclo[3.1.0]hex-6-ylmethyl]}-2-cyclopentyl-2-hydroxy-2-phenyl acetamide (Compound No. 5)
N-{[(1α,5α,6α)-3-(3-Nitrobenzenesulphonyl)-3-azabicyclo[3.1.0]hex-6-ylmethyl]}-2-hydroxy-2-cyclopentyl-2-phenyl acetamide (Compound No. 9)
N-{[(1α,5α,6α)-3-(4-Trifluoromethylbenzenesulfonyl)-3-azabicyclo[3.1.0]-hex-6-ylmethyl]}-2-cyclopentyl-2-hydroxy-2-phenyl acetamide (Compound No. 11)
N-{[(1α,5α,6α)-3-(4-Tert-butylbenzenesulfonyl)-3-azabicyclo[3.1.0]-hex-6-ylmethyl]}-2-cyclopentyl-2-hydroxy-2-phenyl acetamide (Compound No. 14)
N-{[(1α,5α,6α)-3-(4-Fluorobenzenesulphonyl)-3-azabicyclo[3.1.0]hex-6-ylmethyl]}{-2-cyclopentyl-2-hydroxy-2-phenyl acetamide (Compound No. 23)
N-{[(1α,5α,6×)-3-(2,4,6-Trisopropylbenzenesulfonyl)-3-azabicyclo[3.1.0]hex-6-yl methyl]}-2-cyclopentyl-2-hydroxy-2-phenyl acetamide (Compound No. 24)
N-{[(1α,5α,6α)-3-(Methanesulfonyl)-3-azabicyclo[3.1.0]hex-6-yl methyl]}-2-cyclopentyl-2-hydroxy-2-phenyl acetamide (Compound No. 37)
N-{[(1α,5α,6α)-3-(Dimethylsulfamoyl)-3-azabicyclo[3.1.0]hex-6-yl methyl]}-2-cyclopentyl-2-hydroxy-2-phenyl acetamide (Compound No. 41)

### Example 2

### Synthesis of N-{[(1α,5α,6α)-3-[2-(3,5-difluorophenyl)-acetyl]-3-azabicyclo (3.1.0)-hex-6-ylmethyl}-2-cyclopentyl-2-hydroxy-2-phenyl Acetamide (Compound No. 13)

The compound was prepared following the procedure as described for the synthesis of Compound No. 4, Example-1 by using 2,4-difluoro phenyl acetyl chloride in place of p-nitro phenyl sulphonyl chloride with 54% yield; IR (KBr): 1640.9 cm⁻¹; ¹H NMR (CDCl₃): δ 7.59-7.61 (m, 2H), 7.19-7.37 (m, 4H), 6.76-6.86 (m, 2H), 6.65 (brs, 1H), 3.73-3.77 (m, 1H), 3.40-3.51 (m, 4H, including —OH), 3.22-3.23 (m, 7H).

Analogs of N-{[(1α,5α,6α)-3-[2-(3,5-difluorophenyl)-acetyl]-3-azabicyclo-(3.1.0)-hex-6-ylmethyl}-2-hydroxy-2-phenyl acetamide (Compound No. 13) described below, can be prepared by replacing appropriate acyl halide group in place of 2,4-difluoro phenyl acetyl chloride, as applicable in each case.
N-{[(1α,5α,6α)-3-(3,5-Dinitrobenzoyl)-3-azabicyclo[3.1.0]hex-6-ylmethyl]}-2-cyclopentyl-2-hydroxy-2-phenyl acetamide (Compound No. 6),
N-{[(1α,5α,6α)-3-(2-Benzyloxyacetyl)-3-azabicyclo[3.1.0]hex-6-ylmethyl]}-2-cyclopentyl-2-hydroxy-2-phenyl acetamide (Compound No. 7),
N-{[(1α,5α,6α)-3-Benzoyl-3-azabicyclo[3.1.0]hex-6-ylmethyl]}-2-cyclopentyl-2-hydroxy-2-phenyl acetamide (Compound No. 8),
N-{[(1α,5α,6α)-3-(2-Benzo[1,3]dioxol-5-yl-acetyl)-3-azabicyclo[3.1.0]hex-6-ylmethyl]}-2-cyclopentyl-2-hydroxy-2-phenyl acetamide (Compound No. 10),
N-{[(1α,5α,6α)-3-(2-Fluorobenzoyl)-3-azabicyclo[3.1.0]hex-6-ylmethyl]}-2-cyclopentyl-2-hydroxy-2-phenyl acetamide (Compound No. 15),
N-{[(1α,5α,6α)-3-(3,4,5-Trimethoxybenzoyl)-3-azabicyclo[3.1.0]hex-6-ylmethyl]}-2-cyclopentyl-2-hydroxy-2-phenyl acetamide (Compound No. 16),
N-{[(1α,5α,6α)-3-Phenylacetyl-3-azabicyclo[3.1.0]hex-6-ylmethyl]}-2-cyclopentyl-2-hydroxy-2-phenyl acetamide (Compound No. 18),
N-{[(1α,5α,6α)-3-(3,5-Dimethylbenzoyl)-3-azabicyclo[3.1.0]hex-6-ylmethyl]-2-cyclopentyl-2-hydroxy-2-phenyl acetamide (Compound No. 27).

### Example 3

### Synthesis of N-{[(1α,5α,6α)-3-(4-methoxy-benzoyl)-3-azabicyclo[3.1.0]hex-6-yl methyl]}-2-cyclopentyl-2-hydroxy-2-phenyl Acetamide (Compound No. 39)

The compound was synthesised following the procedure as described for the synthesis of compound No. 4, Example-1 by using 4-methoxy benzoyl chloride in place of p-nitro benzene sulfonyl chloride with 90% yield; M.P: 58.3-59.6° C.; IR (KBr): 1656.3, 1610.3 cm⁻¹; ¹H NMR (CDCl₃): δ 7.57-7.60 (m, 2H), 7.21-7.41 (m, 5H), 6.87-6.90 (m, 2H), 3.83 (s, 3H), 3.40-3.59 (m, 3H), 3.03-3.10 (m, 4H), 1.42-1.65 (m, 9H), 1.26-1.40 (m, 2H), 0.72-0.74 (m, 1H); Mass (m/z): 449 (M⁺+1), 431 (M⁺-OH)

Analogs of N-{[(1α,5α,6α)-3-(4-methoxybenzoyl)-3-azabicyclo[3.1.0]hex-6-yl methyl]}-2-cyclopentyl-2-hydroxy-2-phenyl acetamide (Compound No. 39) described below, can be prepared by replacing appropriate acyl halide group in place of 4-methoxy benzoyl chloride as applicable in each case.
N-{[(1α,5α,6α)-3-(3-Benzo[1,3]-dioxol-5-yl-propionyl)-3-azabicyclo[3.1.0]hex-6-yl methyl]}-2-cyclopentyl-2-hydroxy-2-phenyl acetamide (Compound No. 40),

### Scheme I, Path B Procedure

### Example 4

### Synthesis N-{[(1α,5α,6α)-6-[(2-cyclopentyl-2-hydroxy-2-phenyl-acetylamino)-methyl]}-3-aza-bicyclo[3.1.0]hexane-3-carboxylic acid 4-nitro-benzyl ester (Compound No. 19)

The title compound was prepared following the procedure described for the synthesis of Compound No. 4, Example-1 by using 4-nitrobenzyl chloroformate in place of 4-methoxy benzoyl chloride with 46% yield; M.P: 73.1-74.3° C.; IR (KBr): 1635.0 cm⁻¹; ¹H NMR (CDCl₃): δ 7.59-7.62 (m, 2H), 7.29-7.37 (m, 4H), 6.63-6.74 (m, 2H), 6.59 (brs, 1H), 5.91 (s, 2H), 3.72-3.76 (m, 1H), 3.02-3.37 (m, 6H, including —OH), 2.85 (t, 2H, J=6 Hz), 2.42 (t, 2H, J=6 Hz), 1.26-1.66 (m, 11H), 0.75 (m, 1H); Mass (m/z): 491 (M⁺+1), 473 (M⁺-OH).

Analogs of N-{[(1α,5α,6α)-6-[(2-cyclopentyl-2-hydroxy-2-phenyl-acetylamino)-methyl]}-3-aza-bicyclo[3.1.0]hexane-3-carboxylic acid 4-nitro-benzyl ester (Compound no. 19) described below, can be prepared by replacing appropriate chloroformate in place of 4-nitro benzyl chloroformate, as applicable in each case.
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid isobutyl ester (Compound No. 20),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid 4-nitro-phenyl ester (Compound No. 21),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid benzyl ester (Compound No. 22),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid 9H-fluoren-9-ylmethyl ester (Compound No. 35),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid butyl ester (Compound No. 36),

### Scheme II, Path A Procedure

### Example 5

### Synthesis of N-{[(1α,5α,6α)-3-chloro-3-azabicyclo[3.1.01 hex-6-yl methyl]}-2-cyclopentyl-2-hydroxy-2-phenyl acetamide (Compound No. 29)

To a solution of N-(1α,5α,6α)-(3-azabicyclo[3.1.0]hex-6-ylmethyl)-2-cyclopentyl-2-hydroxy-2-phenyl acetamide in dichloromethane (10.0 ml), was added sodium hypochlorite (4.0 ml) at room temperature and the reaction mixture was stirred at room temperature for 3 hours. The reaction mixture was diluted with chloroform and water followed by stirring it for five minutes. The organic layer was separated, washed thoroughly with water, dried and concentrated to get the title organic compound with 90% yield; M.P: 130.7-131.9° C.; IR (KBr): 1656.8 cm⁻¹; ¹H NMR (CDCl₃): δ 7.59-7.61 (m, 2H), 7.30-7.38 (m, 3H), 3.61-3.68 (m, 2H), 3.03-3.17 (m, 4H), 1.49-1.69 (m, 9H), 1.11-1.26 (m, 2H), 0.83 (s, 1H); Mass (m/z): 349 (M⁺+1), 331 (M⁺-OH).

Analogs of N-{[(1α,5α,6α)-3-chloro-3-azabicyclo[3.1.0]hex-6-ylmethyl]}-2-cyclopentyl-2-hydroxy-2-phenyl acetamide (Compound No. 29) described below, can be prepared by replacing appropriate amine in place of (1α,5α,6α)-N-(3-azabicyclo[3.1.0]hex-6-ylmethyl)-2-cyclopentyl-2-hydroxy-2-phenyl-acetamide, as applicable in each case.
N-{[(1α,5α,6α)-3-Chloro-3-azabicyclo[3.1.0]hex-6-ylmethyl]}-2-cyclohexyl-2-hydroxy-2-phenyl acetamide (Compound No. 33),
N-{[(1α,5α,6α)-3-Chloro-3-azabicyclo[3.1.0]hex-6-ylmethyl]}-2-hydroxy-2-diphenyl acetamide (Compound No. 34).

### Example 6

### Synthesis of Hydrochloride Salt of N-{[(1α,5α,6α)-3-chloro-3-azabicyclo[3.1.0]hex-6-ylmethyl]}-2-cyclopentyl-2-hydroxy-2-phenyl Acetamide Hydrochloride Salt (Compound No. 32)

To a solution of the compound No. 29 (0.15 g) in dichloromethane (5.0 ml), was added ethanolic hydrochloric acid solution (3N, 0.5 ml) and stirred the reaction mixture for 10 minutes. The solvent was evaporated off under reduced pressure and the residue thus obtained was triturated with diethylether to get the solid. The solid was dried under vacuum to furnish the title compound with 90% yield; ¹H NMR (DMSO-d₆): 8.02 (t, 1H, J=6 Hz), 7.56-7.59 (m, 2H), 7.19-7.32 (m, 3H), 5.55 (s, 1H), 2.98-3.16 (m, 4H, including —OH), 2.85-2.94 (m, 3H), 1.24-1.53 (m, 11H), 1.07 (t, 1H, J=3 Hz).

### Example 7

### Synthesis of N-{[(1α,5α,6α)-3-cyano-3-azabicyclo[3.1.01 hex-6-ylmethyl]}-2-cyclopentyl-2-hydroxy-2-phenyl acetamide (Compound No. 31)

To a solution of N-(1α,5α,6α)-(3-azabicyclo[3.1.0]hex-1-ylmethyl)-2-cyclopentyl-2-hydroxy-2-phenyl acetamide (0.25 g) and triethylamine (0.22 ml) in dichloromethane (5.0 ml) at room temperature, was added cyanogen bromide (0.25 g) and the reaction mixture was stirred at the same temperature for half on hour. The reaction mixture was cooled to 0° C. followed by the addition of sodium hydroxide (0.5 N) and stirred for 10 minutes. Organic layer was separated, washed with brine solution, dried and concentrated under reduced pressure. The residue was purified by column chromatography using ethylacetate in hexane solvent mixture as an eluent to furnish the title organic compound with 74% yield; M.P: 122.6-123.8° C.; IR (KBr): 2213.5, 1648.4 cm⁻¹; ¹H NMR (CDCl₃): δ 7.59-7.61 (m, 2H), 7.30-7.38 (m, 3H), 6.65 (brs, 1H), 3.32-3.47 (m, 4H, including —OH), 3.09-3.22 (m, 3H), 1.19-1.57 (m, 11H), 0.91 (brs, 1H); Mass (m/z): 340 (M++OH), 322 (M⁺-OH).

### Example 8

### Synthesis of N-{[(1α,5α,6α)-6-{[(2-cyclopentyl-2-hydroxy-2-phenyl-acetylamino)-methyl]}-3-aza-bicyclo[3.1.0]hexane-3-carboxylic acid tert-butyl Ester (Compound No. 1)

To a cold solution of N-(1α,5α,6α)-(3-azabicyclo[3.1.0]hex-1-ylmethyl)-2-cyclopentyl-2-hydroxy acetamide (5.1 mmole) in dichloromethane, was added triethylamine (10.1 mmole) followed by the addition of di-tertbutoxy carbonyl anhydride (6.1 mmole). The reaction mixture was stirred at same temperature for 30 minutes and then at room temperature for 3½ hour. The organic layer was separated and the aqueous layer was extracted with dichloromethane. The combined organic layer was dried over anhydrous sodium sulphate and concentrated. The crude organic compound was purified by column chromatography using ethyl acetate in hexane solvent mixture as eluent to furnish the title compound with 75% yield; ¹H NMR (CDCl₃): δ 7.61-7.59 (2H, m), 7.37-7.28 (3H, m), 6.55 (1H, brs), 3.48-3.45 (2H, m), 3.29-3.26 (2H, m), 3.08-3.03 (3H, m), 1.69-1.55 (8H, m), 1.42-1.36 (9H, m), 1.23-1.18 (2H, m), 0.74-0.72 (1H, m); Mass (m/z): 414 (M⁺+1).

### Scheme II, Path B Procedure

### Example 9

### Synthesis of N-{[(1α,5α,6α)-6-{[(2-cyclopentyl-2-hydroxy-2-phenyl-acetylamino)-methyl]}-3-aza-bicyclo[3.1.0]hexane-3-carboxylic Acid Benzylamide (Compound No. 3)

To a solution of N-(1α,5α,6α)-(3-azabicyclo[3.1.0]hex-1-ylmethyl)-2-cyclopentyl-2-hydroxy-2-phenyl acetamide (0.8 mmole) in dichloroethane (100 ml), was added benzyl isocyanate (0.955 mmole) at room temperature and stirred for 1 hour at the same temperature. The reaction mixture was directly absorbed onto the silica gel and purified by column chromatography using methanol in chloroform as an eluent with 85% yield; m.p: 76-78° C.; IR (KBr): 1636.4, 1527.7 cm⁻¹; ¹H NMR (CDCl₃): δ 7.58-7.61 (m, 2H), 7.23-7.36 (m, 8H), 6.62 (brs, 1H), 4.39 (s, 2H), 3.33-3.47 (m, 4H), 3.06-3.16 (m, 4H), 1.45-1.70 (m, 9H), 1.25 (brs, 2H), 0.79 (t, 1H, J=6 Hz); Mass (m/z): 448 (M⁺+1), 430 (M⁺-OH).

The analogs of N-{[(1α,5α,6α)-6-{[(2-cyclopentyl-2-hydroxy-2-phenyl-acetylamino)-methyl]}-3-aza-bicyclo[3.1.0]hexane-3-carboxylic acid benzylamide (Compound No. 3) described below, can be prepared by replacing appropriate isocyanate in place of benzyl isocyanate, as applicable in each case.
N-{[(1α,5α,6α)-6-{[(2-Cyclopentyl-2-hydroxy-2-phenyl-acetylamino)-methyl]-3-aza-bicyclo[3.1.0]hexane-3-carboxylic acid-(4-trifluoromethyl-phenyl)-amide (Compound No. 12)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-(4-fluoro-phenyl)-amide (Compound No. 17)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid allylamide (Compound No. 25)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-(2,4-dimethoxy-phenyl)-amide (Compound No. 26)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-(4-benzyloxy-phenyl)-amide (Compound No. 28)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid amide (Compound No. 30)
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-(2,4-difluoro-phenyl)-amide (Compound No. 38)

### Scheme III Procedure

### Example 10

### Synthesis of N-{[(1α,5α,6α)-3-terbutyl-carboxy-3-azabicyclo[3.1.0]hex-6-yl methyl]}-2-cyclopentyl-2-methoxy-2-phenyl Acetamide (Compound No. 2)

### Step a: Synthesis of 4-[(2-cyclopentyl-2-hydroxy-2-phenyl acetamide)-methyl]-3-methyl piperidine-1-carboxylic Acid Tert-Butyl Ester

To a solution of (1α,5α,6α)-N-(3-azabicyclo[3.1.0]hex-1-ylmethyl)-2-cyclopentyl-2-hydroxy-2-phenyl acetamide (0.3 g, 1 mmole) in dichloromethane was added trimethyl amine at 0° C. followed by the addition of ditert-butoxy carboxyl anhydride (0.261 g, 1.2 mmole) in dichloromethane. Reaction mixture was stirred at 0° C. for 30 minutes then at room temperature for 3½ hours. The reaction mixture was poured into water and the layers were separated. The aqueous layer was extracted with dichloromethane. The combined organic layer was dried over anhydrous was purified by column chromatography to give the title compound.

### Step b: Synthesis of 4-[(2-cyclopentyl-2-phenyl-2-trimethylaryloxy-acetylamino)-methyl]-3-methyl-piperidine-1-carboxylic Acid Tert-Butyl Ester

To a solution of a compound obtained form step a above (0.414 g, 1 mmole) in dimethylformamide, was added imidazole (0.251 g, 3.7 mmole) followed by the addition of trimethyl silyl chloride (0.293 g, 2.7 mmole) at room temperature and stirred at same temperature for 2 hours. Reaction mixture was poured into water and extracted with diethylether. The organic layer was separated, dried over anhydrous sodium sulphate and concentrated under reduced pressure crude organic compound was purified by column chromatography.

### Step c: Synthesis of N-{[(1α,5α,6α)-3-terbutyl-carboxy-3-azabicyclo[3.1.0]hex-6-yl methyl]}-2-cyclopentyl-2-methoxy-2-phenyl Acetamide (Compound No. 2)

To a solution of a compound obtained from step b above (0.486 g, 1 mmole) in dry tetrahydrofuran at 0° C., was added sodium hydride (0.080 g, 2 mmole, 60% suspension in mineral oil) followed by the addition of tetra n-butyl ammonium iodide (0.025 g, 0.07 mmole). The reaction mixture was stirred at same temperature for 30 minutes and then at room temperature for 1 hour followed by cooling the reaction mixture at 0° C.

To the reaction mixture iodomethane (1.28 g, 9 mmole) was added. The reaction mixture was allowed to warm at room temperature and then stirred overnight. The reaction mixture was quenched with ammonium chloride solution and the organic compound was extracted with ethyl acetate. The organic layer was dried over anhydrous sodium sulphate and concentrated under reduced pressure. The crude organic compound was purified by column chromatography to furnish the title compound with 34% yield; iR (KBr): 1657.8 cm⁻¹; ¹H NMR (CDCl₃): δ 7.26-7.46 (m, 5Ar—H), 6.99 (s, 1H), 3.17-3.25 (m, 2H), 3.15 (s, 3H), 2.87-3.00 (m, 6H), 1.67-1.85 (m, 8H), 0.86-0.88 (m, 2H); Mass (m/z): 428 (M⁺+1).

### Biological Activity

### Radioligand Binding Assays:

The affinity of test compounds for M₂ and M₃ muscarinic receptor subtypes was determined by [³H]-N-methylscopolamine binding studies using rat heart and submandibular gland respectively as described by Moriya et al., (Life Sci, 1999, 64(25):2351-2358) with minor modifications. In competition binding studies, specific binding of [3H]NMS was also determined using membranes from Chinese hamster ovary (CHO) cells expressing cloned human M₁, M₂, M₃, M₄ and M₅ receptors. Selectivities were calculated from the Ki values obtained on these human cloned membranes.

Membrane preparation: Submandibular glands and heart were isolated and placed in ice cold homogenising buffer (HEPES 20 mM, 10 mM EDTA, pH 7.4) immediately after sacrifice. The tissues were homogenised in 10 volumes of homogenising buffer and the homogenate was filtered through two layers of wet gauze and filtrate was centrifuged at 500 g for 10 min. The supernatant was subsequently centrifuged at 40,000 g for 20 min. The pellet thus obtained was resuspended in assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μl of assay volume (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The IC₅₀ & Kd were estimated by using the non-linear curve-fitting program using G Pad Prism software. The value of inhibition constant Ki was calculated from competitive binding studies by using Cheng & Prusoff equation (Biochem Pharmacol, 1973, 22: 3099-3108), Ki=IC₅₀/(1+L/Kd), where L is the concentration of [³H]NMS used in the particular experiment. pki is −log [Ki].

### Functional Experiments Using Isolated Rat Bladder: Methodology:

Animals were euthanized by overdose of thiopentone and whole bladder was isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder was cut into longitudinal strips (3 mm wide and 5-6 mm long) and mounted in 10 ml organ baths at 30° C., with one end connected to the base of the tissue holder and the other end connected through a force displacement transducer. Each tissue was maintained at a constant basal tension of 1 g and allowed to equilibrate for 1½ hour during which the Tyrode buffer was changed every 15-20 min. At the end of equilibration period the stabilization of the tissue contractile response was assessed with 1 μmol/L of Carbachol till a reproducible response is obtained. Subsequently a cumulative concentration response curve to carbachol (10⁻⁹ mol/L to 3×10⁻⁴ mol/L) was obtained. After several washes, once the baseline was achieved, cumulative concentration response curve was obtained in presence of NCE (NCE added 20 min. prior to the second cumulative response curve.

The contractile results were expressed as % of control E max. ED₅₀ values were calculated by fitting a non-linear regression curve (Graph Pad Prism). pK_{b} values were calculated by the formula pK_{b}=−log [(molar concentration of antagonist/(dose ratio−1))] where, dose ratio=ED₅₀ in the presence of antagonist/ED₅₀ in the absence of antagonist. The results of in-vitro tests are found to be ≦10.

The particular compounds specified herein exhibited Kᵢ values for M₂ receptors of from about 10,000 nM to about 7.8 nM, for example from about 1000 nM to about 7.8 nM, or from about 60 nM to about 7.8 nM, or from about 9.0 to about 7.8 nM. The particular compounds specified herein exhibited Kᵢ values for M₃ receptors of from about 1000 nM to about 0.5 nM, for example from about 500 nM to about 0.5 nM, or from about 30 nM to about 0.5 nM, or from about 0.7 to about 0.5 nM.

While the present invention has been described in terms of its specific embodiments, certain modification and equivalents will be apparent to those skilled in the art and are intended to be included within the scope of the present invention.

We claim:

1. Compounds having the structure of Formula I 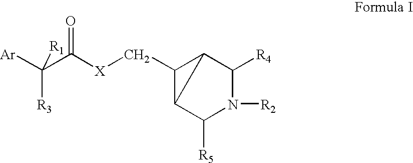
and its pharmaceutically acceptable salts, pharmaceutically acceptable solvates, stereoisomers or polymorphs, wherein
R₁ is hydrogen, lower (C₁-C₆) alkyl, lower (C₂-C₇) alkenyl, lower (C₂-C₇) alkynyl, cycloalkyl, amino, substituted amino, —OR_{z} {wherein R_{z} is selected from hydrogen, —Si(CH₃)₃, lower (C₁-C₆) alkyl, lower (C₂-C₆) alkenyl, lower (C₂-C₆) alkynyl, cycloalkyl, aryl, and —C(═O)NHRᵣ (wherein Rᵣ is selected from hydrogen, lower (C₁-C₆) alkyl, lower (C₂-C₆) alkenyl, lower (C₂-C₆) alkynyl, aryl, and cycloalkyl)};
R₂ is carboxy, —SO₂R₆ {wherein R₆ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, and heteroarylalkyl), aryl, aralkyl, heteroaryl, heterocyclyl, heterocyclylalkyl, and heteroarylalkyl, or Rₚ and R_{q} may also together join to form a heterocyclyl ring}, —C(═O)OR₇ (wherein R₇ is selected from alkyl, alkenyl, alkynyl, cycloalkyl, aryl, and aralkyl), —C(═O)NRₓR_{y} (wherein Rₓ and R_{y} are each independently selected from hydrogen, hydroxy (as restricted by the definition that both Rₓ and R_{y} cannot be hydroxy at the same time), alkyl, alkenyl, alkynyl, aryl, aralkyl, S(O)₂R₆ wherein R₆ is the same as defined above, heteroaryl, heterocyclyl, heteroarylalkyl, and heterocyclylalkyl, or Rₓ and R_{y} may also together join to form a heterocyclyl ring), acyl, halogen (F, Cl, Br, I), cyano, —NRₓR_{y}, wherein Rₓ and R_{y} are the same as defined above), or —C(═O)CH₂ORₓ (wherein Rₓ is the same as defined above);
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
X is oxygen, —NR₇ (wherein R₇ is selected from hydrogen, lower (C₁-C₆) alkyl, lower (C₂-C₇) alkenyl, lower (C₂-C₇) alkynyl, aralkyl, and aryl; and
Ar is aryl, heteroaryl, and heterocyclyl.

2.-3. (canceled)

4. A compound according to claim 1, wherein Ar is phenyl.

5.-6. (canceled)

7. A compound according to claim 1, wherein R₁ is —OR_{z} and R_{z} is hydrogen.

8. A compound according to claim 1, wherein R₁ is —OR_{z} and R_{z} is alkyl.

9.-12. (canceled)

13. A compound according to claim 1, wherein R₇ is tert-butyl, isobutyl, fluorinemethyl, isopropyl or butyl.

14. (canceled)

15. A compound according to claim 1, wherein R₂ is —C(═O)NRₓR_{y}.

16.-19. (canceled)

20. A compound according to claim 1, wherein R_{y} is fluorophenyl, trifluoromethylphenyl, difluorophenyl, dimethoxyphenyl or benzyloxyphenyl.

21.-27. (canceled)

28. A compound according to claim 1, wherein R₂ is —C(═O)CH₂ORₓ.

29.-30. (canceled)

31. A compound according to claim 1, wherein R₂ is cyano.

32. A compound according to claim 1, wherein R₂ is halogen.

33. (canceled)

34. A compound according to claim 1, wherein R₂ is acyl.

35.-46. (canceled)

47. A compound selected from
N-{[(1α,5α,6α)-6-{[(2-Cyclopentyl-2-hydroxy-2-phenyl-acetylamino)-methyl]-3-aza-bicyclo[3.1.0]hexane-3-carboxylic acid tert-butyl ester (Compound No. 1),
N-{[(1α,5α,6α)-3-Terbutyl-carboxy-3-azabicyclo[3.1.0]hex-6-ylmethyl]}-2-cyclopentyl-2-methoxy-2-phenyl acetamide (Compound No. 2),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid benzylamide (Compound No. 3),
N-{[(1α,5α,6α)-3-(4-Nitrobenzenesulphonyl)-3-azabicyclo[3.1.0]hex-6-ylmethyl]}-2-cyclopentyl-2-hydroxy-2-phenyl acetamide (Compound No. 4),
N-{[(1α,5α,6α)-(3,5-Benzenesulfonyl-3-azabicyclo[3.1.0]hex-6-ylmethyl]}-2-cyclopentyl-2-hydroxy-2-phenyl acetamide (Compound No. 5),
N-{[(1α,5α,6α)-3-(3,5-Dinitrobenzoyl)-3-azabicyclo[3.1.0]hex-6-ylmethyl]}-2-cyclopentyl-2-hydroxy-2-phenyl acetamide (Compound No. 6),
N-{[(1α,5α,6α)-3-(2-Benzyloxyacetyl)-3-azabicyclo[3.1.0]hex-6-ylmethyl])-2-cyclopentyl-2-hydroxy-2-phenyl acetamide (Compound No. 7),
N-{[(1α,5α,6α)-3-Benzoyl-3-azabicyclo[3.1.0]hex-6-ylmethyl]}-2-hydroxy-2-cyclopentyl-2-phenyl acetamide (Compound No. 8),
N-{[(1α,5α,6α)-3-(3-Nitrobenzenesulphonyl)-3-azabicyclo[3.1.0]hex-6-ylmethyl]}-2-hydroxy-2-cyclopentyl-2-phenyl acetamide (Compound No. 9),
N-{[(1α,5α,6α)-3-(2-Benzo[1,3]dioxol-5-yl-acetyl)-3-azabicyclo[3.1.0]hex-6-ylmethyl]}-2-hydroxy-2-cyclopentyl-2-phenyl acetamide (Compound No. 10),
N-{[(1α,5α,6α)-3-(4-Trifluoromethylbenzenesulfonyl)-3-azabicyclo[3.1.0]hex-6-ylmethyl]}-2-cyclopentyl-2-hydroxy-2-phenyl acetamide (Compound No. 11),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (4-trifluoromethyl-phenyl)-amide (Compound No. 12),
N-{[(1α,5α,6α)-3-[2-(3,5-Difluoro-phenyl)-acetyl]-3-azabicyclo[3.1.0]hex-6-ylmethyl]}-2-hydroxy-2-cyclopentyl-2-phenyl acetamide (Compound No. 13),
N-{[(1α,5α,6α)-3-(4-Tert-butylbenzenesulfonyl)-3-azabicyclo[3.1.0]hex-6-ylmethyl]}-2-cyclopentyl-2-hydroxy-2-phenyl acetamide (Compound No. 14),
N-{[(1α,5α,6α)-3-(2-Fluorobenzoyl)-3-azabicyclo[3.1.0]hex-6-ylmethyl}]-2-cyclopentyl-2-hydroxy-2-phenyl acetamide (Compound No. 15),
N-{[(1α,5α,6α)-3-(3,4,5-Trimethoxybenzoyl)-3-azabicyclo[3.1.0]hex-6-yl methyl]}-2-cyclopentyl-2-hydroxy-2-phenyl acetamide (Compound No. 16),
N-{[(1α,5α,6α)-6-{[(2-Cyclopentyl-2-1hydroxy-2-phenyl-acetylamino)-methyl]}-3-aza-bicyclo[3.1.0]hexane-3-carboxylic acid (4-fluorophenyl)-amide (Compound No. 17),
N-{[(1α,5α,6α)-3-Phenylacetyl-3-azabicyclo[3.1.0]hex-6-ylmethyl]}-2-cyclopentyl-2-hydroxy-2-phenyl acetamide (Compound No. 18),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid-4-nitro-benzyl ester (Compound No. 19),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid isobutyl ester (Compound No. 20),
N-{[(1α,5α,6α)-6-{[(2-Cyclopentyl-2-hydroxy-2-phenyl-acetylamino)-methyl-3-aza-bicyclo[3.1.0]hexane-3-carboxylic acid 4-nitro-phenyl ester (Compound No. 21),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid benzyl ester (Compound No. 22),
N-{[(1α,5α,6α)-3-(4-Fluorobenzenesulphonyl)-3-azabicyclo[3.1.0]hex-6-ylmethyl)}]-2-cyclopentyl-2-hydroxy-2-phenyl acetamide (Compound No. 23),
N-{[(1α,5α,6α)-3-(2,4,6-Trisopropylbenzenesulphonyl)-3-azabicyclo[3.1.0]hex-6-yl methyl]}-2-cyclopentyl-2-hydroxy-2-phenyl acetamide (Compound No. 24),
N-{[(1α,5α,6α)-6-[(2-Cyclopentyl-2-hydroxy-2-phenyl-acetylamino)-methyl]}-3-aza-bicyclo[3.1.0]hexane-3-carboxylic acid allylamide (Compound No. 25),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (2,4-dimethoxy-phenyl)-amide (Compound No. 26),
N-{[(1α,5α,6α)-3-(3,5-Dimethylbenzoyl)-3-azabicyclo[3.1.0]hex-6-ylmethyl]}-2-cyclopentyl-2-hydroxy-2-phenyl acetamide (Compound No. 27),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (4-benzyloxy-phenyl)-amide (Compound No. 28),
N-{[(1α,5α,6α)-3-Chloro-3-azabicyclo[3.1.0]hex-6-ylmethyl]}-2-cyclopentyl-2-hydroxy-2-phenyl acetamide (Compound No. 29),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid amide (Compound No. 30),
N-{[(1α,5α,6α)-3-Cyano-3-azabicyclo[3.1.0]hex-6-ylmethyl]}-2-cyclopentyl-2-hydroxy-2-phenyl acetamide (Compound No. 31),
N-{[(1α,5α,6α)-3-Chloro-3-azabicyclo[3.1.0]hex-6-ylmethyl]}-2-cyclopentyl-2-hydroxy-2-phenyl acetamide hydrochloride salts (Compound No. 32),
N-{[(1α,5α,6α)-3-Chloro-3-azabicyclo[3.1.0]hex-6-ylmethyl]}-2-cyclohexyl-2-hydroxy-2-phenyl acetamide (Compound No. 33),
N-{[(1α,5α,6α)-3-Chloro-3-azabicyclo[3.1.0]hex-6-ylmethyl]}-2-hydroxy-2-diphenyl acetamide (Compound No. 34),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid 9H-fluoren-9-ylmethyl ester (Compound No. 35),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid butyl ester (Compound No. 36),
N-{[(1α,5α,6α)-3-(Methanesulphonyl)-3-azabicyclo[3.1.0]hex-6-ylmethyl]}-2-cyclopentyl-2-hydroxy-2-phenyl acetamide (Compound No. 37),
N-{[(1α,5α,6α)-6-{[(2-Cyclopentyl-2-hydroxy-2-phenyl-acetylamino)-methyl]}-3-aza-bicyclo[3.1.0]hexane-3-carboxylic acid (2,4-difluoro-phenyl)-amide (Compound No. 38),
N-{[(1α,5α,6α)-3-(4-Methoxybenzoyl)-3-azabicyclo[3.1.0]hex-6-ylmethyl]}-2cyclopentyl-2-hydroxy-2-phenyl acetamide (Compound No. 39),
N-{[(1α,5α,6α)-3-(3-Benzo[1,3]-dioxol-5-yl-propionyl)-3-azabicyclo[3.1.0]hex-6-yl methyl]}-2-cyclopentyl-2-hydroxy-2-phenyl acetamide (Compound No. 40),
N-{[(1α,5α,6α)-3-(Dimethylsulfamoyl)-3-azabicyclo[3.1.0]hex-6-yl methyl]}-2-cyclopentyl-2-hydroxy-2-phenyl acetamide (Compound No. 41).
their pharmaceutically acceptable salts, pharmaceutically acceptable solvates, stereoisomers or polymorphs.

48. A pharmaceutical composition comprising a therapeutically effective amount of a compound as defined in claim 1 together with pharmaceutically acceptable carriers, excipients or diluents.

49. A method for treatment or prophylaxis of an animal or a human suffering from a disease or disorder of the respiratory, urinary and gastrointestinal systems, wherein the disease or disorder is mediated through muscarinic receptors, comprising administering to said animal or human, a therapeutically effective amount of a compound having the structure of Formula I of claim 1.

50. The method according to claim 49, wherein the disease or disorder is urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

51. (canceled)

52. A process of preparing a compound of Formula VI 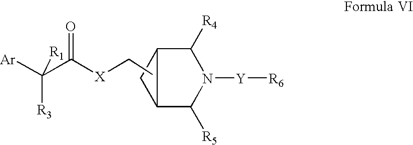
and its pharmaceutically acceptable salts, pharmaceutically acceptable solvates, stereoisomers or polymorphs,
wherein
R₁ is hydrogen, lower (C₁-C₆) alkyl, lower (C₂-C₇) alkenyl, lower (C₂-C₇) alkynyl, cycloalkyl, amino, substituted amino, —OR_{z} {wherein R_{z} is selected from hydrogen, —Si(CH₃)₃, lower (C₁-C₆) alkyl, lower (C₂-C₆) alkenyl, lower (C₂-C₆) alkynyl, cycloalkyl, aryl, and —C(═O)NHRᵣ (wherein Rᵣ is selected from hydrogen, lower (C₁-C₆) alkyl, lower (C₂-C₆) alkenyl, lower (C₂-C₆) alkynyl, aryl, and cycloalkyl)};
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
R₆ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, and heteroaryalkyl), aryl, aralkyl, heteroaryl, heterocyclyl, heterocyclylalkyl, and heteroarylalkyl, or Rₚ and R_{q} may also together join to form a heterocyclyl ring;
X is oxygen, —NR₇ (wherein R₇ is selected from hydrogen, lower (C₁-C₆) alkyl, lower (C₂-C₇) alkenyl, lower (C₂-C₇) alkynyl, aralkyl, and aryl; and
Ar is aryl, heteroaryl, and heterocyclyl,
the process comprising:
a) condensing a compound of Formula II (wherein Ar, R₁ and R₃ are the same as defined earlier) with a compound of Formula III (wherein X, R₄ and R₅ are the same as defined earlier and P is a protecting group) to give a compound of Formula IV, 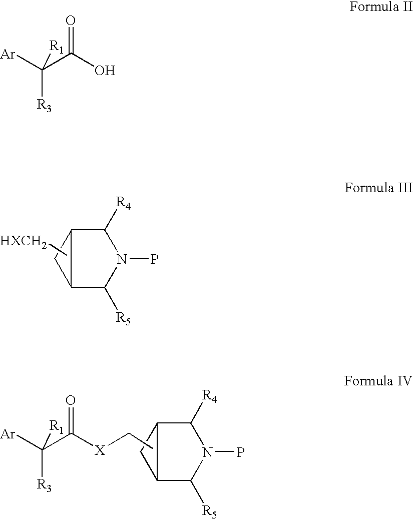
b) deprotecting the compound of Formula IV to give a compound of Formula V, and 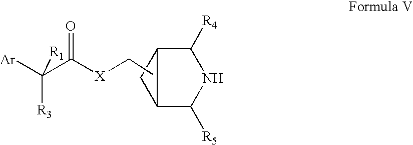
c) reacting the compound of Formula V with a compound of Formula L-Y—R₆ (wherein L is a leaving group, Y is —C(═O), SO₂ and R₆ is the same as defined earlier) to give a compound of Formula VI.

53. A process of preparing a compound of Formula VII 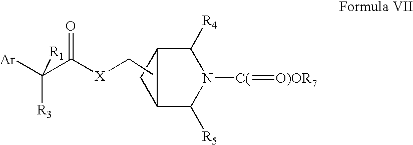
wherein
R₁ is hydrogen, lower (C₁-C₆) alkyl, lower (C₂-C₇) alkenyl, lower (C₂-C₇) alkynyl, cycloalkyl, amino, substituted amino, —OR_{z} {wherein R_{z} is selected from hydrogen, —Si(CH₃)₃, lower (C₁-C₆) alkyl, lower (C₂-C₆) alkenyl, lower (C₂-C₆) alkynyl, cycloalkyl, aryl, and —C(═O)NHRᵣ (wherein Rᵣ is selected from hydrogen, lower (C₁-C₆) alkyl, lower (C₂-C₆) alkenyl, lower (C₂-C₆) alkynyl, aryl, and cycloalkyl)};
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
R₆ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, and heteroarylalkyl), aryl, aralkyl, heteroaryl, heterocyclyl, heterocyclylalkyl, and heteroarylalkyl, or R_{P} and R_{q} may also together join to form a heterocyclyl ring;
R₇ is selected from hydrogen, lower (C₁-C₆) alkyl, lower (C₂-C₇) alkenyl, lower (C₂-C₇) alkynyl, aralkyl, and aryl;
X is oxygen, —NR₇ (wherein R₇ is as defined above); and
Ar is aryl, heteroaryl, and heterocyclyl,
the process comprising:
a) condensing a compound of Formula II (wherein Ar, R₁ and R₃ are the same as defined earlier) with a compound of Formula III (wherein X, R₄ and R₅ are the same as defined earlier and P is a protecting group) to give a compound of Formula IV, 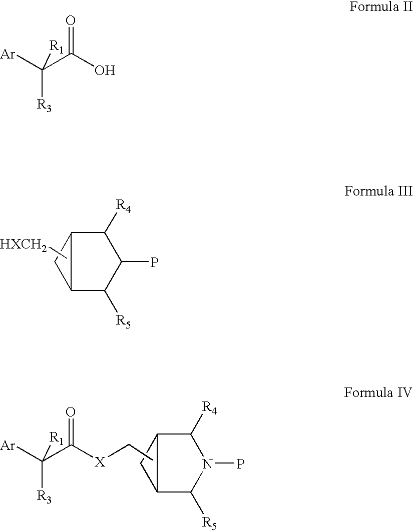
b) deprotecting the compound of Formula IV to give a compound of Formula V, and 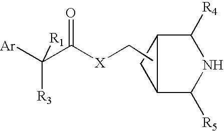
reacting the compound of Formula V with a compound of Formula
hal-C(═O)OR₇ (wherein R₇ is the same as defined earlier and hal is halogen) to give a compound of Formula VII.

54. A process of preparing a compound of Formula IX 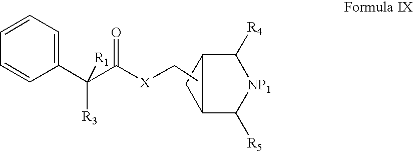
and its pharmaceutically acceptable salts, pharmaceutically acceptable solvates, stereoisomers or polymorphs, wherein.
R₁ is hydrogen, lower (C₁-C₆) alkyl, lower (C₂-C₇) alkenyl, lower (C₂-C₇) alkynyl, cycloalkyl, amino, substituted amino, —OR_{z} {wherein R_{z} is selected from hydrogen, —Si(CH₃)₃, lower (C₁-C₆) alkyl, lower (C₂-C₆) alkenyl, lower (C₂-C₆) alkynyl, cycloalkyl, aryl, and —C(═O)NHRᵣ (wherein Rᵣ is selected from hydrogen, lower (C₁-C₆) alkyl, lower (C₂-C₆) alkenyl, lower (C₂-C₆) alkynyl, aryl, and cycloalkyl)};
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
R₇ is selected from hydrogen, lower (C₁-C₆) alkyl, lower (C₂-C₇) alkenyl, lower (C₂-C₇) alkynyl, aralkyl, and aryl;
X is oxygen, or —NR₇ (wherein R₇ is as defined above); and
P₁ is halogen (F, Cl, Br or I), cyano or —C(═O)OR₇ (R₇ is the same as defined earlier)
the process comprising:
N-protecting the compound of Formula VIII to give a compound of Formula IX 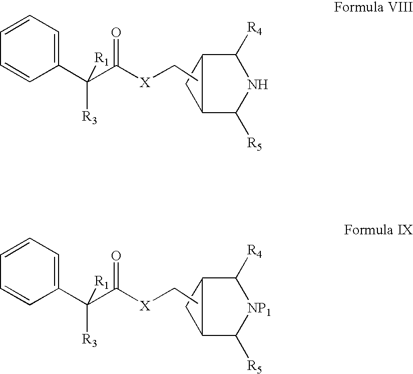
[wherein P₁ is halogen (F, Cl, Br or I), cyano or —C(═O)OR₇ (R₇ is the same as defined earlier)].

55. A process of preparing a compound of Formula XI 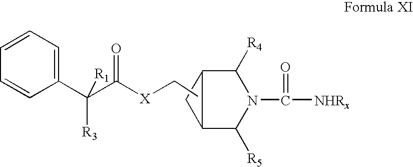
wherein,
R₁ is hydrogen, lower (C₁-C₆) alkyl, lower (C₂-C₇) alkenyl, lower (C₂-C₇) alkynyl, cycloalkyl, amino, substituted amino, —OR_{z} {wherein R_{z} is selected from hydrogen, —Si(CH₃)₃, lower (C₁-C₆) alkyl, lower (C₂-C₆) alkenyl, lower (C₂-C₆) alkynyl, cycloalkyl, aryl, and —C(═O)NHRᵣ (wherein Rᵣ is selected from hydrogen, lower (C₁-C₆) alkyl, lower (C₂-C₆) alkenyl, lower (C₂-C₆) alkynyl, aryl, and cycloalkyl)};
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
X is oxygen, or —NR₇ (wherein R₇ is as defined above); and
Rₓ is hydrogen, hydroxy, alkyl, alkenyl, alkynyl, aryl, aralkyl, S(O)₂R₆ {wherein R₆ is R₆ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, and heteroarylalkyl), aryl, aralkyl, heteroaryl, heterocyclyl, heterocyclylalkyl, and heteroarylalkyl, or Rₚ and R_{q} may also together join to form a heterocyclyl ring}, heteroaryl, heterocyclyl, heteroarylalkyl, and heterocyclylalkyl,
the process comprising
reacting a compound of Formula VIII with a compound of Formula X 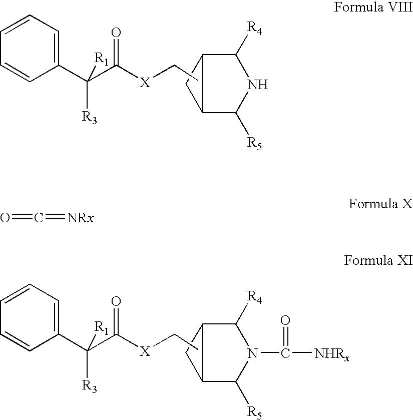
(wherein Rₓ is the same as defined earlier) to give a compound of Formula XI.

56. A process of preparing a compound of Formula XIV 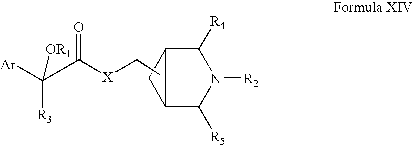
and its pharmaceutically acceptable salts, pharmaceutically acceptable solvates, stereoisomers or polymorphs, wherein,
R₂ is carboxy, —SO₂R₆ {wherein R₆ is selected from alkyl, alkenyl, alkynyl, cycloalkyl, —NRₚR_{q} (wherein Rₚ and R_{q} are selected from hydrogen, alkyl, alkenyl, alkynyl, cycloalkyl, aryl, aralkyl, heterocyclyl, heteroaryl, heterocyclylalkyl, and heteroarylalkyl), aryl, aralkyl, heteroaryl, heterocyclyl, heterocyclylalkyl, and heteroarylalkyl, or Rₚ and R_{q} may also together join to form a heterocycyl ring), —C(═O)OR₇ (wherein R₇ is selected from alkyl, alkenyl, alkynyl, cycloalkyl, aryl, and aralkyl), —C(═O)NRₓR_{y} (wherein Rₓ and R_{y} are each independently selected from hydrogen, hydroxy (as restricted by the definition that both Rₓ and R_{y} cannot be hydroxy at the same time), alkyl, alkenyl, alkynyl, aryl, aralkyl, S(O)₂R₆ wherein R₆ is the same as defined above, heteroaryl, heterocyclyl, heteroarylalkyl, and heterocyclylalkyl, or Rₓ and R_{y} may also together join to form a heterocyclyl ring), acyl, halogen (F, Cl, Br, I), cyano, —NRₓR_{y}, wherein Rₓ and R_{y} are the same as defined above), or —C(═O)CH₂ORₓ (wherein Rₓ is the same as defined above);
R₃ is alkyl, alkenyl, alkynyl, cycloalkyl, aryl, aralkyl, heterocyclyl, heteroaryl, heterocyclylalkyl, and heteroarylalkyl;
R₄ and R₅ are independently selected from hydrogen, lower (C₁-C₆) alkyl, lower (C₂-C₇) alkenyl, and lower (C₂-C₇) alkynyl;
Rₜ is alkyl;
X is oxygen, —NR₇ (wherein R₇ is selected from hydrogen, lower (C₁-C₆) alkyl, lower (C₂-C₇) alkenyl, lower (C₂-C₇) alkynyl, aralkyl, and aryl; and
Ar is aryl, heteroaryl, and heterocyclyl
which comprises:
a) reacting a compound of Formula XII with trimethyl silyl chloride to give a compound of Formula XIII, and 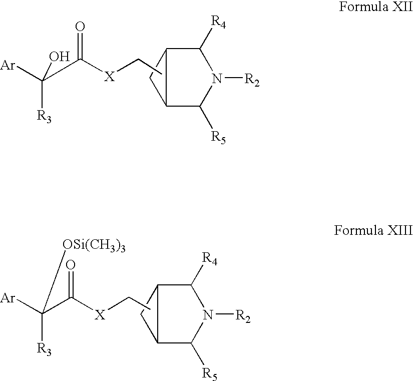
b) O-alkylating the compound of Formula XIII to give a compound of Formula XIV.
